(19) United States
(12) Patent Application Publication
(10) Pub. No.: US 2026/0174950 A1
(43) Pub. Date: Jun. 25, 2026

(54) Tamper-resistant cuff, and a system and a user pack, for the storage and retrieval of a sterile disposable injection needle

(71) Applicant: Michael Anthony Martinez, San Francisco, CA (US)

(72) Inventor: Michael Anthony Martinez, San Francisco, CA (US)

(21) Appl. No.: 18/990,402

(22) Filed: Dec. 20, 2024

(51) Int. Cl. A61M 5/00; A61M 5/32; A61M 5/34

(52) U.S. Cl. CPC A61M 5/002; A61M 5/3202; A61M 5/34; A61M 2209/06

(57) ABSTRACT

A system for a storage and retrieval of a sterile disposable injection needle having a needle end and a syringe female connector end, comprising: the sterile disposable injection needle; a needle cap, the needle cap having a needle cap closed distal end and a needle cap open proximal end for snugly receiving the sterile disposable injection needle; and a needle cap cuff for encircling the needle cap open proximal end, the cuff comprising a cylindrical ring with a cuff distal end and a cuff proximal end; wherein when the cuff distal end encircles the needle cap open proximal end, the cuff proximal end is positioned at least a first height above the syringe female connector end of the sterile disposable injection needle, such that the cuff inhibits or prevents a user from accessing the needle that is positioned in the needle cap and that is detached from a syringe.

### CROSS-REFERENCE TO RELATED APPLICATIONS STATEMENTS REGARDING FEDERALLY SPONSORED RESEARCH AND DEVELOPMENT

Not applicable.

### BACKGROUND

The embodiments of the present invention satisfy the needs of having a better tamper-resistant device and system for the storage and retrieval of a sterile disposable injection needle.

Information relevant to attempts to address these problems can be found in U.S. Pat. Nos. 10,682,475, 7,654,981, and 5,347,078; which are not admitted to be prior art with respect to the present invention by their mention in this Background Section. It is desirable to have better apparatuses and/or methods than what is disclosed in the identified references.

U.S. Pat. No. 10,682,475 discloses and claims a small container with a passageway for receiving a needle. The syringe is pushed into the passageway and snapped into place by means of ribs or detents at the proximal rim of the passageway. The needle-tipped cap can then be unscrewed from the syringe. There is one passageway for an unused needle and a second passageway for a used needle. The passageway for receiving the used needle comprises a lip at the top to prevent removal of the needle once it is snapped in, rendering it tamper-resistant and irreversibly positioned such that it cannot be removed even with a syringe.

U.S. Pat. No. 7,654,981 discloses and claims a passageway for receiving a needle-tipped syringe. The distal end of the passageway comprises a conical friction fit opening that accepts the needle and then holds it in place while the user unscrews or otherwise removes the syringe. The user then pushes the needle down and out of the friction fit. The device can be placed on the top of a box to collect the used needles. The device is not tamper-resistant.

U.S. Pat. No. 5,347,078 discloses and claims a receptacle having a trap door or flaps over a passageway for receiving a needle-tipped syringe, where the distal end of the passageway comprises a cylindrical friction fit opening that accepts the needle-tipped cap and then holds it in place while the user unscrews or otherwise removes the syringe from it. The trap door or flaps prevent removal of the needle from the container, rendering it tamper-resistant and irreversible such that it cannot be removed.

These aforementioned innovations require a separate container for carrying an unused needle; namely, they require a container separate from the needle cap in which the needle was originally provided. Two of these innovations are tamper-resistant but they are irreversible, which further prevents their use as containers for unused needles, i.e., a user must remove the needle from the original needle cap for its use, use the needle, and then subsequently dispose of the needle in the claimed container. Patent '475 seeks to address that issue by providing a first passageway for holding the unused needle and a second passageway for holding the used needle, but this requires more plastic material which is expensive and which creates more waste. Thus there is a need for a system for the storage and retrieval of a sterile disposable injection needle into and from the needle cap in which the needle was originally provided, and doing so in a manner such that a stored needle is tamper-resistant.

### SUMMARY

The embodiments of the present invention comprise a cuff for the storage and retrieval of a sterile disposable injection needle in a sterile disposable injection needle cap, the sterile disposable injection needle having a distal needle end and a proximal syringe female connector end, the sterile disposable injection needle cap having a distal closed end and a proximal open end, the cuff comprising a cylindrical ring with a cuff distal end and a cuff proximal end; wherein the sterile disposable injection needle cap is adapted for snugly receiving the sterile disposable injection needle; wherein when the cuff distal end encircles the sterile disposable injection needle cap proximal open end, the cuff proximal end is positioned at least a first height above the syringe female connector end of the sterile disposable injection needle, thereby inhibiting or preventing a user from accessing the sterile disposable injection needle when it is detached from a syringe and positioned in the sterile disposable injection needle cap.

The cuff replaces the trap door and detents of previous innovations. It has the benefit of providing tamper resistance while still allowing for reversibility.

Optionally, the sterile disposable injection needle cap comprises an outwardly extending needle cap abutment rib encircling an outer periphery of the needle cap, and the cuff distal end comprises a plurality of cantilever snap fit joints for forming a snap fit with the needle cap abutment rib.

Optionally, the cuff monolithically extends from the needle cap open end.

Optionally, the first height is between 0 to 14 millimeters, inclusive.

Optionally, the first height is between 3 and 3.5 millimeters, inclusive.

The embodiments of the present invention further comprise a system for a storage and retrieval of a sterile disposable injection needle having a needle end and a syringe female connector end, comprising: the sterile disposable injection needle with the needle end and with the syringe female connector end; a needle cap, the needle cap having a needle cap closed distal end and a needle cap open proximal end for snugly receiving the sterile disposable injection needle; and a needle cap cuff for encircling the needle cap open proximal end, the cuff comprising a cylindrical ring with a cuff distal end and a cuff proximal end; wherein when the cuff distal end encircles the needle cap open proximal end, and the cuff proximal end is positioned at least a first height above the syringe female connector end of the sterile disposable injection needle, such that the cuff inhibits or prevents a user from accessing the needle that is positioned in the needle cap and that is detached from a syringe.

Optionally, the system further comprises a stand for the needle cap closed distal end, wherein the stand enables a user to insert the sterile disposable injection needle into the needle cap without holding the needle cap in hand.

Optionally, the stand comprises a base portion and a pedestal portion, wherein a proximal end of the pedestal portion is open for receiving the needle cap closed distal end.

Optionally, the stand base portion is disk-shaped.

Optionally, the sterile disposable injection needle cap comprises an outwardly extending needle cap abutment rib encircling an outer periphery of the needle cap, and the cuff distal end comprises a plurality of cantilever snap fit joints for forming a snap fit with the needle cap abutment rib.

Optionally, the needle cap cuff monolithically extends from the needle cap open end.

Optionally, the stand monolithically extends from the needle cap distal end.

Optionally, an exterior of the sterile disposable injection needle comprises fins, and an interior of the needle cap comprises ridges for engaging the fins, such that the injection needle when positioned in the needle cap is screwably detachable from a syringe.

Optionally, the first height is between 0 to 14 millimeters, inclusive.

Optionally, the first height is between 3 and 3.5 millimeters, inclusive.

An embodiment of the present invention comprises a user pack containing at least one component selected from a sterile disposable injection needle cap comprising an outwardly extending needle cap abutment rib encircling an outer periphery of the needle cap, and a needle cap cuff having distal end comprising a plurality of cantilever snap fit joints for forming a snap fit with the needle cap abutment rib; and an information insert containing directions on the use of both components together in combination.

Optionally, the needle cap abutment rib extends monolithically from the exterior of the needle cap.

An embodiment of the present invention comprises a user pack containing at least one component selected from a sterile disposable injection needle cap comprising an outwardly extending needle cap abutment rib encircling an outer periphery of the needle cap, and a needle cap cuff having distal end comprising a plurality of cantilever snap fit joints for forming a snap fit with the needle cap abutment rib, and a stand for the needle cap; and an information insert containing directions on the use of the three components together in combination.

Optionally, the needle cap abutment rib extends monolithically from the exterior of the needle cap.

### Some Advantages of the Embodiments of the Invention

The cuff has a benefit of providing tamper resistance while still allowing for reversibility, such that the original needle cap for the unused needle can be used to dispose of the needle in a tamper-resistant manner.

The cuff is tamper-resistant as it prevents the removal of a needle without a tool or syringe, reducing potential sticks and declassifying its medical waste classification.

The stand removes the hand from holding the cap and being in the direct line of the needle when capping, preventing sticks.

These and other advantages will become apparent as the embodiments of the invention are used.

### DESCRIPTIONS OF THE DRAWINGS

These and other features, aspects and advantages of the embodiments of the device and/or methods will become better understood with reference to the following description, appended claim and accompanying drawings where:

FIG. 1 shows a front elevation of an embodiment of the cuff, wherein the back, right, and left elevations are substantially the same;

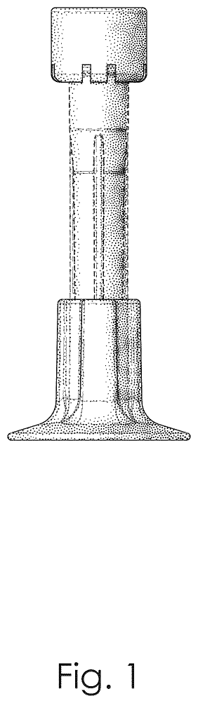

FIG. 2 shows a top plan view of an embodiment of the cuff;

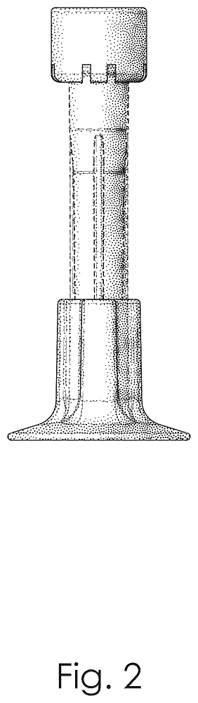

FIG. 3 shows a bottom plan view of an embodiment of the cuff;

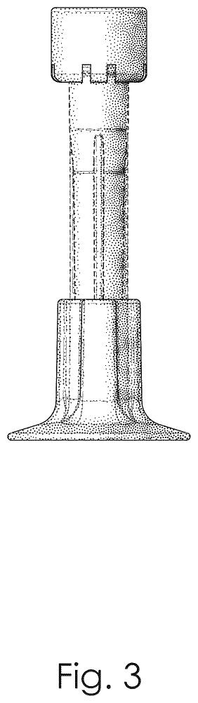

FIG. 4 shows front elevation of an embodiment of the stand, wherein the back, right, and left elevations are substantially the same;

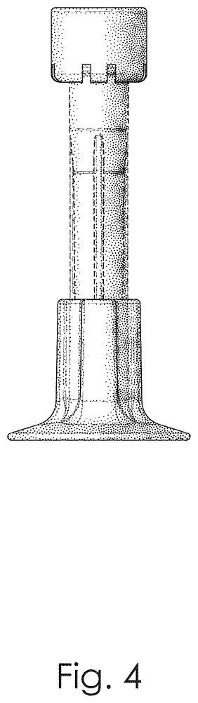

FIG. 5 shows a top plan view of an embodiment of the stand;

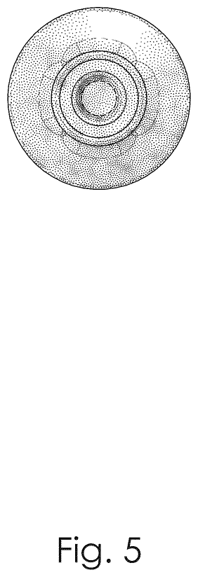

FIG. 6 shows a bottom plan view of an embodiment of the stand;

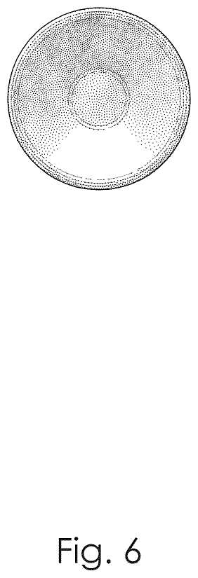

FIG. 7 shows an embodiment of the present invention in which the needle cap comprises a needle cap lip encircling an exterior of the needle cap, and the cuff distal end comprises a plurality of cantilever snap fit joints for forming an irreversible snap fit with the needle cap lip;

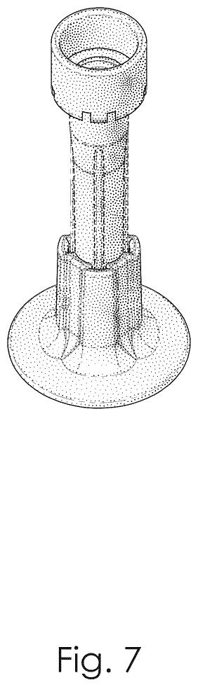

FIG. 8 shows an embodiment of the present invention in which the needle cap cuff monolithically extends from the needle cap open end;

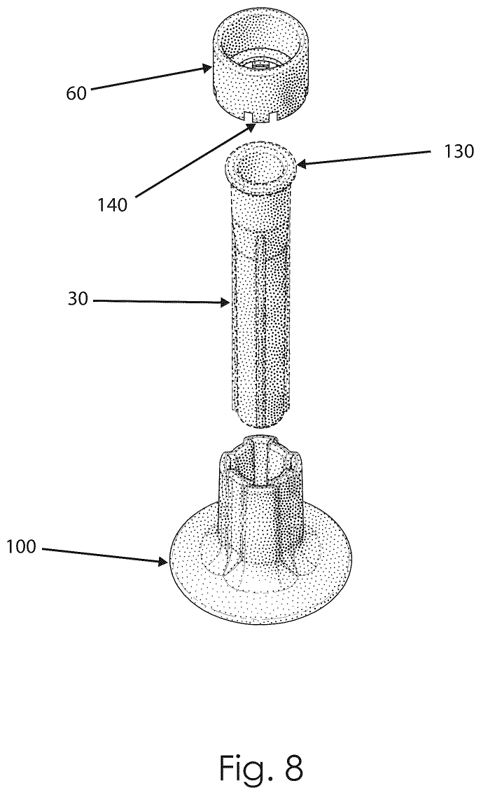

FIG. 9a shows an embodiment of the present invention in which the stand monolithically extends from the needle cap distal end;

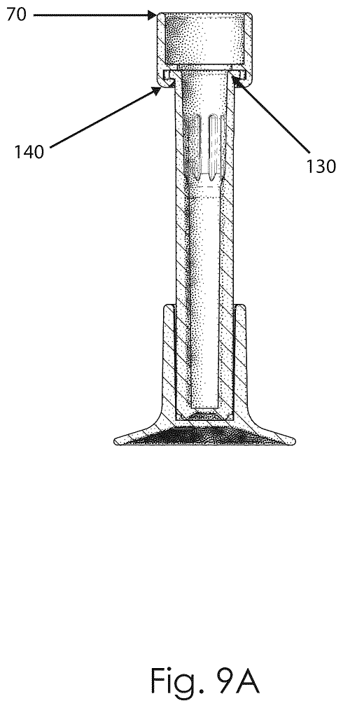

FIG. 9b shows an embodiment of the present invention in which the stand monolithically extends from the needle cap distal end, and the needle is inserted into the needle cap; and

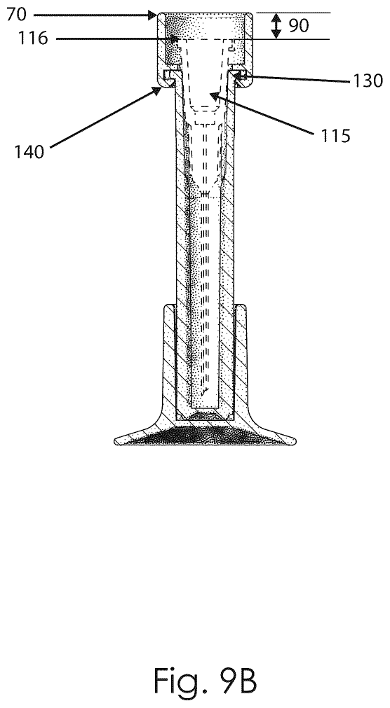

FIG. 10 shows an embodiment of the present invention in which an exterior of the sterile disposable injection needle comprises fins, and an interior of the needle cap comprises ridges for engaging the fins, such that the injection needle when positioned in the needle cap is screwably detachable from a syringe.

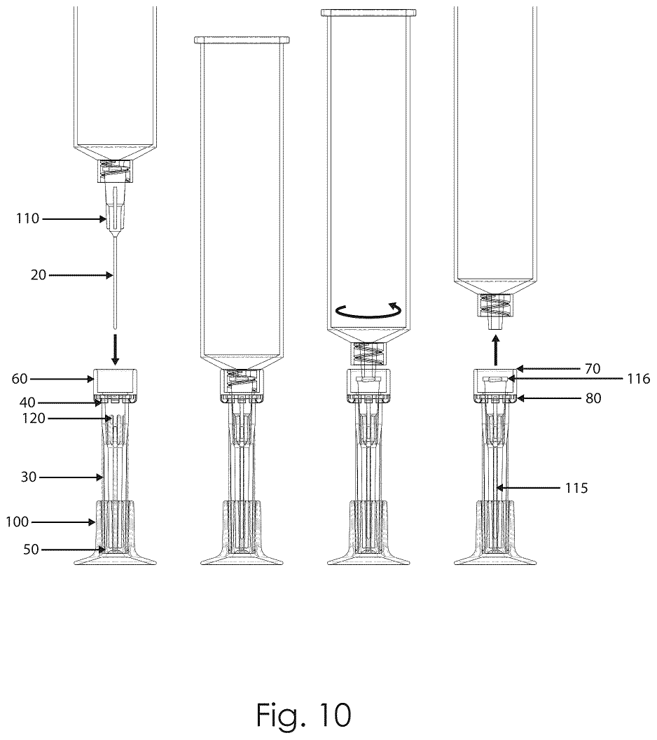

### REFERENCE NUMERALS FOR DRAWINGS

20 sterile disposable injection needle
30 needle cap
40 needle cap open (proximal) end
50 needle cap closed distal end
60 needle cap cuff
70 cuff proximal end
80 cuff distal end
90 first height
100 stand
110 fins
115 sterile disposable injection needle
116 syringe female connector end
120 ridges
130 needle cap abutment rib
140 cantilever snap fit joint

### DESCRIPTION

### Definitions

A cuff is often defined as something, such as a part of a sleeve or glove or shackle or other wide band, encircling the wrist. Here we define a cuff not for a wrist but instead for a sterile disposable injection needle. Like a wrist cuff, the cuff contemplated herein also encircles, but it encircles the wider open end of a sterile injection needle cap.

A sterile disposable injection needle cap comprises an open end and a closed end. A sterile disposable injection needle comprises a needle end and an open syringe female connector end for receiving a syringe male connector. This open syringe female connector end may also comprise a Luer lock, which is an outwardly extending needle cap abutment rib encircling an outer periphery of the connector end. A snap fit between a needle and a needle cap is accomplished by a specialized needle cap with grooves that lock the cap in place to preserve sterility of the syringe.

As shown in the figures, the preferred embodiments and best mode of using the present invention comprise a system for a storage and retrieval of a sterile disposable injection needle, comprising: a sterile disposable injection needle; a needle cap, the needle cap having a needle cap closed distal end and a needle cap open proximal end for snugly receiving the sterile disposable injection needle; and a needle cap cuff for encircling the needle cap open proximal end, the cuff comprising a cylindrical ring with a cuff distal end and a cuff proximal end; wherein when the cuff distal end encircles the needle cap open proximal end, and the cuff proximal end is positioned at least a first height above the needle cap open end, such that the cuff inhibits or prevents a user from accessing the needle that is positioned in the needle cap and that is detached from a syringe.

Preferably, the system further comprises a stand for the needle cap closed distal end, wherein the stand enables a user to insert the sterile disposable injection needle into the needle cap without holding the needle cap in hand.

Preferably, the stand comprises a base portion and a pedestal portion, wherein a proximal end of the pedestal portion is open for receiving the needle cap closed distal end.

Preferably, the stand base portion is disk-shaped.

Optionally, the needle cap comprises an outwardly extending needle cap abutment rib encircling an outer periphery of the needle cap, and the cuff distal end comprises a plurality of cantilever snap fit joints for forming a snap fit with the needle cap abutment rib. But preferably, the needle cap cuff monolithically extends from the needle cap open end.

Preferably, the stand monolithically extends from the needle cap distal end.

Optionally, an exterior of the sterile disposable injection needle comprises fins, and an interior of the needle cap comprises ridges for engaging the fins, such that the injection needle when positioned in the needle cap is screwably detachable from a syringe.

Optionally, the first height is between 0 to 14 millimeters, inclusive.

Preferably, the first height is between 3 and 3.5 millimeters, inclusive.

The user inserts the needle cap into the stand, and then inserts the syringe-mounted needle into the needle cap, and then twists off the needle from the syringe. In some cases, the needle snap-fits into the needle cap. In some cases, an exterior of the sterile disposable injection needle comprises fins, and an interior of the needle cap comprises ridges for engaging the fins, such that the injection needle when positioned in the needle cap is screwably detachable from a syringe.

Some advantages of the embodiments of the apparatus were previously enumerated in the Summary section. Every advantageous feature does not need to be incorporated into every embodiment of the apparatus and/or methods.

Although these versions of the invention have been described in considerable detail, other versions are possible. For example, embodiments can comprise combinations of the features described herein, such as combinations of the dependent claims. Therefore, the spirit and scope of the appended claims should not be limited to the description of the versions contained therein.

I claim:

1. A cuff for a tamper-resistant storage and retrieval of a sterile disposable injection needle in a sterile disposable injection needle cap, the sterile disposable injection needle having a distal needle end and a proximal syringe female connector end, the sterile disposable injection needle cap having a distal closed end and a proximal open end, the cuff comprising a cylindrical ring with a cuff distal end and a cuff proximal end; wherein the sterile disposable injection needle cap is adapted for snugly receiving the sterile disposable injection needle; wherein when the cuff distal end encircles the sterile disposable injection needle cap proximal open end, the cuff proximal end is positioned at least a first height above the syringe female connector end of the sterile disposable injection needle when it is snugly disposed in the needle cap, thereby inhibiting or preventing a user from accessing the sterile disposable injection needle therein and creating tamper-resistant storage; such that a user can retrieve the sterile disposable injection needle when it is snugly disposed in the needle cap by inserting a syringe male connector into the proximal syringe female connector end.

2. The cuff of claim 1, wherein the sterile disposable injection needle cap comprises an outwardly extending needle cap abutment rib encircling an outer periphery of the needle cap, and the cuff distal end comprises a plurality of cantilever snap fit joints for forming a snap fit with the needle cap abutment rib.

3. The cuff of claim 1, wherein the cuff monolithically extends from the needle cap open end.

4. The cuff of claim 1, wherein the first height is between 0 and 14 millimeters, inclusive.

5. The cuff of claim 4, wherein the first height is between 3 and 3.5 millimeters, inclusive.

6. A system for a storage and retrieval of a sterile disposable injection needle having a needle end and a syringe female connector end, comprising: the sterile disposable injection needle with the needle end and with the syringe female connector end; a needle cap, the needle cap having a needle cap closed distal end and a needle cap open proximal end for snugly receiving the sterile disposable injection needle; and a needle cap cuff for encircling the needle cap open proximal end, the cuff comprising a cylindrical ring with a cuff distal end and a cuff proximal end; wherein when the cuff distal end encircles the needle cap open proximal end, and the cuff proximal end is positioned at least a first height above the syringe female connector end of the sterile disposable injection needle, such that the cuff inhibits or prevents a user from accessing the needle that is positioned in the needle cap and that is detached from a syringe.

7. The system of claim 6, further comprising a stand for the needle cap closed distal end, wherein the stand enables a user to insert the sterile disposable injection needle into the needle cap without holding the needle cap in hand.

8. The system of claim 7, wherein the stand comprises a base portion and a pedestal portion, wherein a proximal end of the pedestal portion is open for receiving the needle cap closed distal end.

9. The system of claim 8, wherein the stand base portion is disk-shaped.

10. The system of claim 6, wherein the sterile disposable injection needle cap comprises an outwardly extending needle cap abutment rib encircling an outer periphery of the needle cap, and the cuff distal end comprises a plurality of cantilever snap fit joints for forming a snap fit with the needle cap abutment rib.

11. The system of claim 6, wherein the needle cap cuff monolithically extends from the needle cap open end.

12. The system of claim 7, wherein the stand monolithically extends from the needle cap distal end.

13. The system of claim 6, wherein an exterior of the sterile disposable injection needle comprises fins, and an interior of the needle cap comprises ridges for engaging the fins, such that the injection needle when positioned in the needle cap is screwably detachable from a syringe.

14. The system of claim 6, wherein the first height is between 0 and 14 millimeters, inclusive.

15. The system of claim 14, wherein the first height is between 3 and 3.5 millimeters, inclusive.

16. A user pack containing at least one component selected from a sterile disposable injection needle cap comprising an outwardly extending needle cap abutment rib encircling an outer periphery of the needle cap, and a needle cap cuff having distal end comprising a plurality of cantilever snap fit joints for forming a snap fit with the needle cap abutment rib; and an information insert containing directions on the use of both components together in combination.

17. The user pack of claim 16, wherein the needle cap abutment rib extends monolithically from the exterior of the needle cap.

18. A user pack containing at least one component selected from a sterile disposable injection needle cap comprising an outwardly extending needle cap abutment rib encircling an outer periphery of the needle cap, and a needle cap cuff having distal end comprising a plurality of cantilever snap fit joints for forming a snap fit with the needle cap abutment rib, and a stand for the needle cap; and an information insert containing directions on the use of the three components together in combination.

19. The user pack of claim 18, wherein the needle cap abutment rib extends monolithically from the exterior of the needle cap.
